(19) United States
(12) Patent Application Publication
(10) Pub. No.: US 2025/0218601 A1
(43) Pub. Date: Jul. 3, 2025

(54) Assessing Health Effects of Landscape Designs

(71) Applicants: Huaqing Wang, West Haven, UT (US); Louis G. Tassinary, Iowa City, IA (US)

(72) Inventors: Huaqing Wang, West Haven, UT (US); Louis G. Tassinary, Iowa City, IA (US)

(73) Assignee: Utah State University, Logan, UT (US)

(21) Appl. No.: 18/400,061

(22) Filed: Dec. 29, 2023

(51) Int. Cl. G16H 50/80; G06N 5/022

(52) U.S. Cl. CPC G16H 50/80; G06N 5/022

(57) ABSTRACT

For predicting a non-communicable disease score for a given landscape design map, a method extracts design characteristics from at least one landscape design map. The method further extracts non-communicable disease data for adjacent populations to the at least one landscape design map. The method trains a predictive model based on the design characteristics and the non-communicable disease data. The method predicts a non-communicable disease for the given landscape design map from the predictive model.

### BACKGROUND INFORMATION

The subject matter disclosed herein relates to assessing health effects of landscape designs.

### BRIEF DESCRIPTION

A method for predicting a non-communicable disease score for a given landscape design map is disclosed. The method extracts design characteristics from at least one landscape design map. The method further extracts non-communicable disease data for adjacent populations to the at least one landscape design map. The method trains a predictive model based on the design characteristics and the non-communicable disease data. The method predicts a non-communicable disease for a given landscape design map from the predictive model. An apparatus and computer program product also perform the elements of the method.

### BRIEF DESCRIPTION OF DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic block diagram illustrating one embodiment of an assessment system;

FIG. 2A is a schematic block diagram illustrating one embodiment of assessment data;

FIG. 2B is a schematic block diagram illustrating one embodiment of design characteristics;

FIG. 2C is a schematic block diagram illustrating one embodiment of greenspace and morphology data;

FIG. 2D is a schematic block diagram illustrating one embodiment of non-communicable disease data 205;

FIG. 3A is an image of a landscape design map;

FIG. 3B is an image of extracted design characteristics;

FIGS. 4A-F are images of relative landscape metrics for greenspace and morphology data;

FIG. 5 is a schematic block diagram of one embodiment of a training process;

FIG. 6 is a schematic block diagram of one embodiment of a computer;

FIG. 7A is a schematic flow chart diagram of an assessment method; and

FIG. 7B is a schematic flow chart diagram of an implementation method.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. The term “and/or” indicates embodiments of one or more of the listed elements, with “A and/or B” indicating embodiments of element A alone, element B alone, or elements A and B taken together.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

The computer readable medium may be a tangible computer readable storage medium storing the program code. The computer readable storage medium may be, for example, but not limited to, an electronic, magnetic, optical, electromagnetic, infrared, holographic, micromechanical, or semiconductor system, apparatus, or device, or any suitable combination of the foregoing.

More specific examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store program code for use by and/or in connection with an instruction execution system, apparatus, or device.

Program code for carrying out operations for aspects of the present invention may be written in any combination of one or more programming languages, including an object-oriented programming language such as Matlab, Python, Ruby, R, Java, Java Script, Julia, Smalltalk, C++, C sharp, Lisp, Clojure, PHP or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). The computer program product may be shared, simultaneously serving multiple customers in a flexible, automated fashion.

The schematic flowchart diagrams and/or schematic block diagrams in the Figures illustrate the architecture, functionality, and operation of possible implementations. It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Although various arrow types and line types may be employed in the flowchart and/or block diagrams, they are understood not to limit the scope of the corresponding embodiments. Indeed, some arrows or other connectors may be used to indicate only an exemplary logical flow of the depicted embodiment.

The description of elements in each figure may refer to elements of proceeding figures. Like numbers refer to like elements in all figures, including alternate embodiments of like elements.

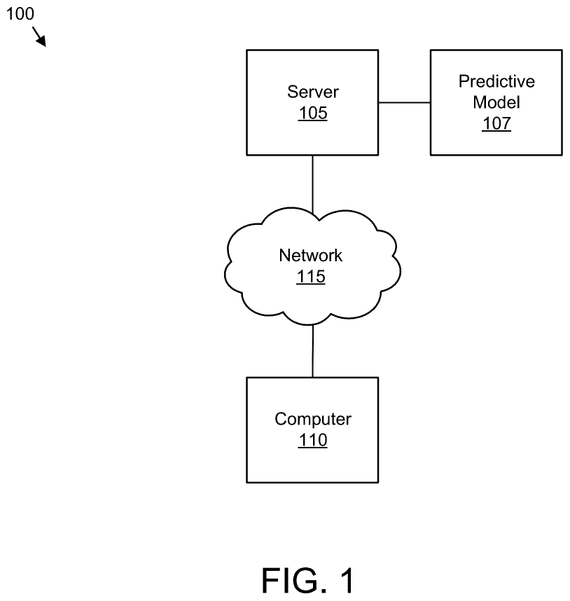

FIG. 1 is a schematic block diagram illustrating one embodiment of an assessment system 100. The assessment system 100 may train a predictive model 107 based on design characteristics and non-communicable disease data. In addition, the assessment system 100 may generate and/or select a landscape design map for implementations. In the depicted embodiment, the assessment system 100 includes a server 105, a predictive model 107, a computer 110, and/or a network 115. In the depicted embodiment, the computer 110 communicates with the predictive model 107 through the server 105. Alternatively, the computer 110 may access and/or communicate directly with the predictive model 107.

In one embodiment, the computer 110 receives a landscape design map. The computer 110 may extract the design characteristics from the landscape design map. In addition, the computer 110 may extract non-communicable disease data for the landscape design map.

Alternatively, the computer 110 may communicate the landscape design map to the server 105 and the server 105 may extract the design characteristics and the non-communicable disease data.

The server 105 and/or computer 110 may train the predictive model 107. The predictive model 107 may be trained based on the design characteristics and non-communicable disease data.

In one embodiment, the predictive model 107 is used to predict a non-communicable disease score for a given landscape design map. The non-communicable disease score may be used to improve the utility of a landscape design and/or landscape design map. As a result, the assessment system 100 improves the efficiency and efficacy of designing a landscape design map. In addition, the assessment system 100 may improve the efficiency and efficacy of the computer and/or server 105.

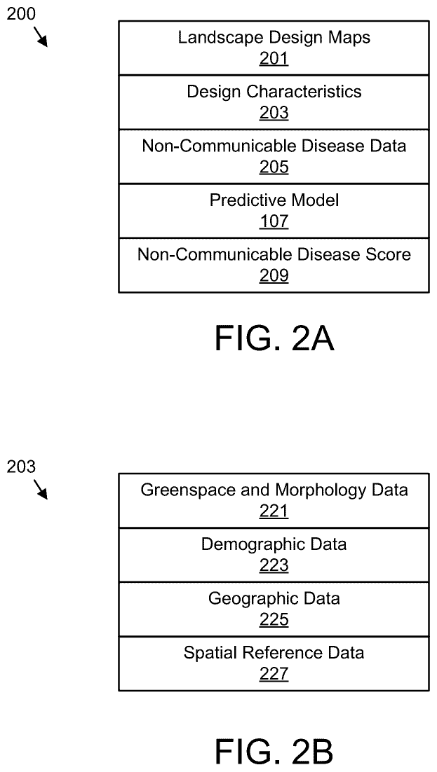

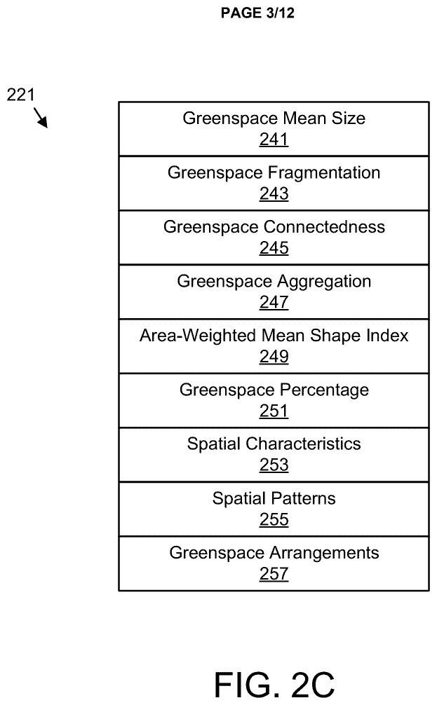

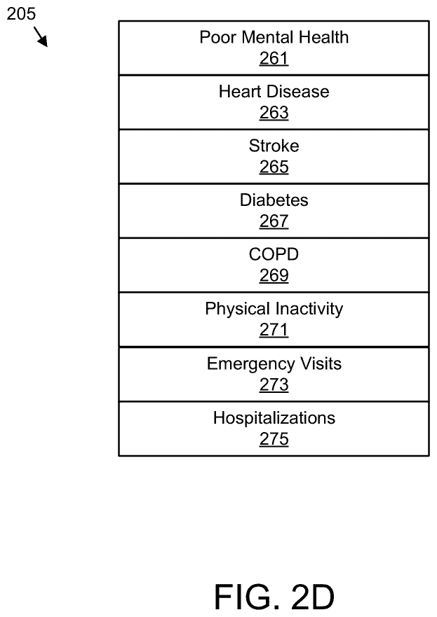

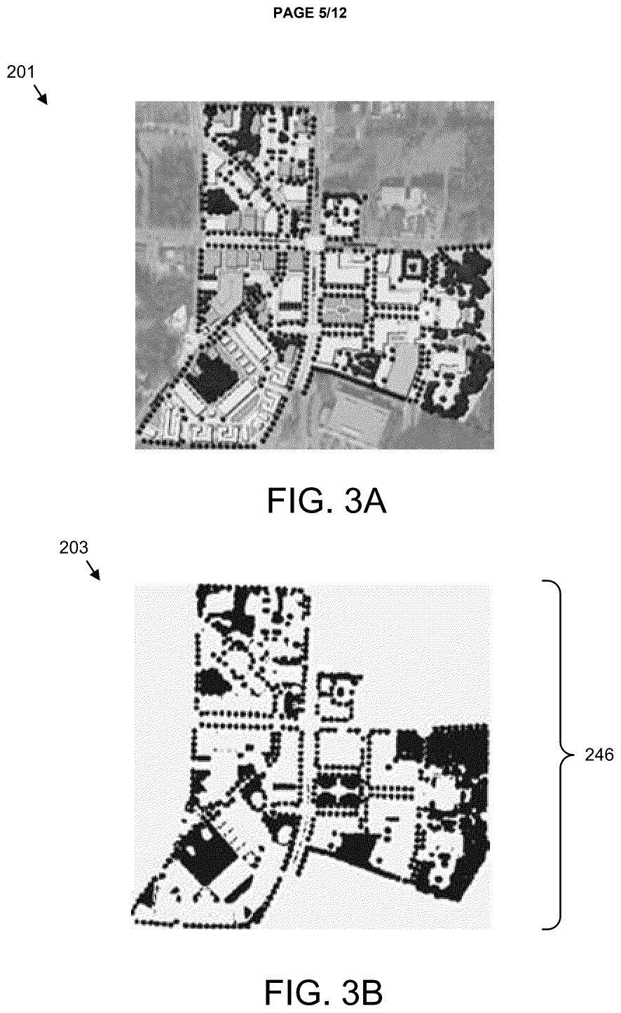

FIG. 2A is a schematic block diagram illustrating one embodiment of assessment data 200. The assessment data 200 may be organized as a data structure in a memory. In the depicted embodiment, the assessment data 200 includes landscape design maps 201, design characteristics 203, non-communicable disease data 205, the predictive model 107, and the non-communicable disease score 209.

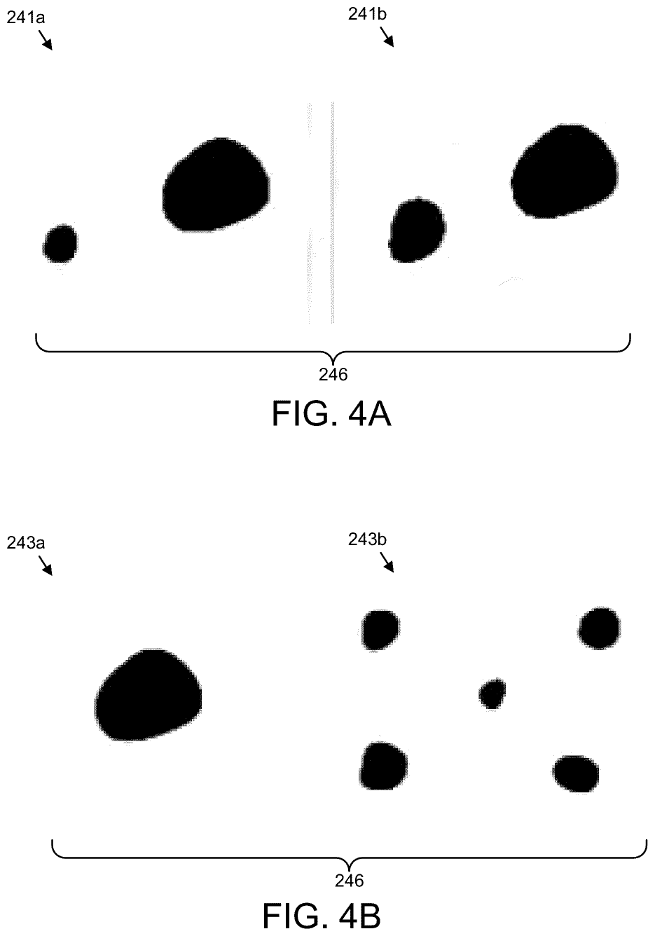

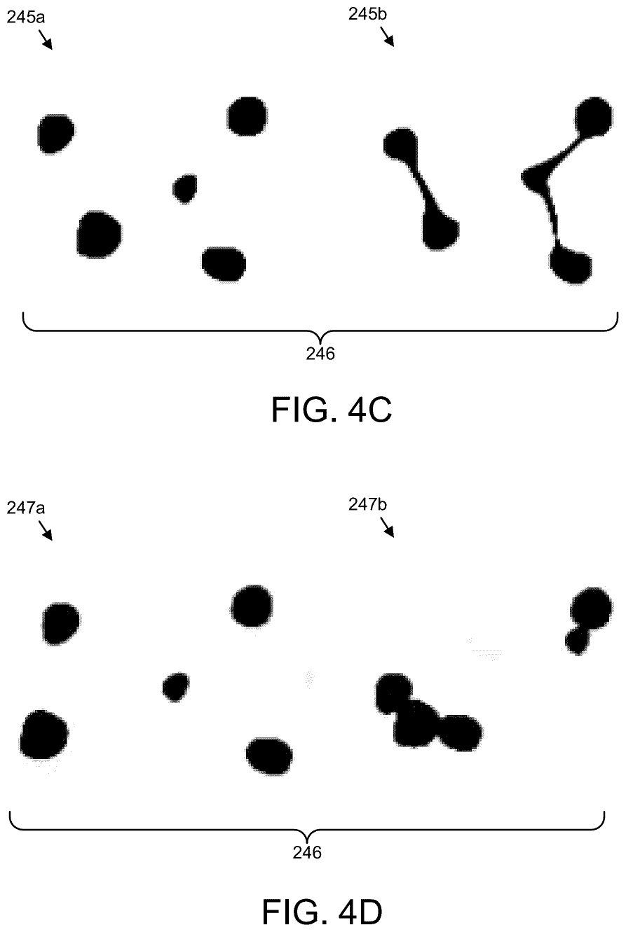

Each landscape design map 201 may describe a landscape design for a specified location. The landscape design map 201 may be encoded in at least one computer file such as the computer-aided design (CAD) file. Alternatively, the landscape design map 201 may be scanned into at least one computer file from a physical drawing. An exemplary landscape design map 201 is shown hereafter in FIG. 3A.

The design characteristics 203 may be extracted from the landscape design map 201. In one embodiment, a subset of landscape design map features are extracted from the landscape design map 201. In a certain embodiment, geographic data from a CAD file are used to reference additional information such as demographic data. For example, the geographic data may be used to reference the non-communicable disease data 205, hospital data, postal code data, and the like. The postal code data may be used to download the non-communicable disease data 205, hospital data, and/or demographic data. One embodiment of design characteristics 203 is described in FIG. 2B.

The non-communicable disease data 205 may describe the prevalence of non-communicable diseases for populations adjacent to the landscape design. In one embodiment, the adjacent population is within a design boundary of the landscape design site limit.

Alternatively, the adjacent population may be within an adjacent postal code. Hospital data may be accessed for hospitals within a hospital buffer of the landscape design. One embodiment of non-communicable disease data 205 is described in FIG. 2C.

The non-communicable disease score 209 provides a quantitative and/or qualitative prediction of the effect of a landscape design embodied in the landscape design map 201 on the health of residents adjacent to the implemented landscape design. The use of non-communicable disease data 205 for the adjacent population improves the efficacy of landscape design maps 201 beyond the capabilities of a human designer. As a result, the non-communicable disease score 209 improves the efficacy and/or efficiency of a landscape design process.

FIG. 2B is a schematic block diagram illustrating one embodiment of the design characteristics 203. In the depicted embodiment, the design characteristics 203 include greenspace and morphology data 221, demographic data 223, geographic data 225, and spatial reference data 227.

The greenspace and morphology data 221 may describe the greenspace and the greenspace morphology of greenspaces in the landscape design. As used herein, greenspace refers to the portion of the landscape design map 201 that includes vegetation. In one embodiment, greenspace is completely covered in vegetation. The greenspace may be comprised of at least one patch.

Alternatively, greenspace may be partially covered by at least one plant with space between the plants. In a certain embodiment, greenspace may be covered by a specified type of vegetation. For example, greenspace may refer to an area comprising at least one of grass, groundcover, shrubs, flowers, and/or trees. The greenspace and morphology data 221 may be organized to improve the efficiency and/or efficacy of calculating the non-communicable disease score 209. The greenspace and morphology data 221 is described in more detail in FIG. 2C.

The demographic data 223 may describe the numbers, gender, race, income, and ages of the adjacent population to the landscape design. In addition, the demographic data 223 may describe the prevalence of non-communicable diseases among the population. The demographic data 223 may divide the adjacent population by age, such older than 65 and not yet 65. In addition, the demographic data 223 may divide the adjacent population into genders. In one embodiment, the demographic data 223 divides the adjacent population by education. In one embodiment, the demographic data 223 includes a population size. In addition, the demographic data 223 may include a population density.

The geographic data 225 include centroid coordinates for features in the landscape design map 201 and/or the adjacent population.

FIG. 2C is a schematic block diagram illustrating one embodiment of the greenspace and morphology data 221. In one embodiment, greenspace morphology is calculated based on a Euclidean buffered area of the landscape design. The Euclidean buffer may be in the range of 0.25 to 0.75 miles. In a certain embodiment, the Euclidean buffer is 0.5 miles. In the depicted embodiment, the greenspace and morphology data 221 includes a greenspace mean size 241, a greenspace fragmentation 243, a greenspace connectedness 245, a greenspace aggregation 247, an area weighted mean shape index 249, a greenspace percentage 251, spatial characteristics 253, spatial patterns 255, and greenspace arrangements 257.

The greenspace mean size 241 quantifies the mean of the greenspace area in the landscape design map 201. The greenspace mean size 241 may be calculated using metric AREA_MN in Table 1. In an alternative embodiment, the greenspace mean size 241 quantifies the average of the greenspace area in the landscape design map 201.

TABLE 1

| Metric | Formula | |
|---|---|---|
| PD | $PD = \frac{{n}_{i}}{A} ( 10000 ) ( 100 )$ | nᵢ = number of patches in the landscape patch i. A = total landscape area (m²). |
| AREA__MN | ${x}_{ij} = {a}_{ij} ( \frac{1}{10000} ) , AREA\_MN = \frac{{\sum }_{j = 1}^{n} {x}_{ij}}{{n}_{i}}$ | aᵢⱼ = area (m²) of patch ij. MN (Mean) equals the sum, across all patches of the patch |
| | | type, of the patch metric values, |
| | | divided by the number of |
| | | patches of the same type. MN is |
| | | given in the same units as the |
| | | corresponding patch metric. |
| COHESION | $COHESION = ( 1 - \frac{{\sum }_{j = 1}^{n} {p}_{ij}}{{\sum }_{j = 1}^{n} {p}_{ij} \sqrt{{a}_{ij}}} ) {( 1 - \frac{1}{\sqrt{A}} )}^{- 1} ( 100 )$ | pᵢⱼ = perimeter of patch ij in terms of the number of cell surfaces. aᵢⱼ = area of patch ij in terms of |
| | | the number of cells. |
| | | A = total number of cells in the |
| | | landscape. |
| AI | $AI = ( \frac{{g}_{ii}}{max ( {g}_{ii} )} ) ( 100 )$ | gᵢᵢ = the number of like adjacencies (joins) between |
| | | pixels of type (class) i based on |
| | | the single-count method. |
| | | max (gᵢᵢ) = the maximum |
| | | number of like adjacencies |
| | | (joins) between pixels of patch |
| | | type (class) I based on the |
| | | single-count method. |
| SHAPE__AM | $SHAPE = \frac{{p}_{ij}}{min ( {p}_{ij} )} , AM = {\sum }_{j = 1}^{n} [ {x}_{ij} ( \frac{{a}_{ij}}{{\sum }_{j = 1}^{n} {a}_{ij}} ) ]$ | pᵢⱼ = perimeter of patch ij in terms of the number of cell surfaces. min(pᵢⱼ) = minimum perimeter |
| | | of patch ij in terms of the |
| | | number of cell surfaces. |
| | | AM (area-weighted mean) |
| | | equals the sum, across all |
| | | patches of the corresponding |
| | | patch type, of the corresponding |
| | | patch metric value multiplied |
| | | by the proportional abundance |
| | | of the patch i.e., patch area (m²) |
| | | divided by the sum of patch |
| | | areas. |
| PLAND | $PLAND = \frac{{\sum }_{j = 1}^{n} {a}_{ij}}{A} * 100$ | aᵢⱼ = the area of each patch. A = the total landscape area. |

The greenspace fragmentation 243 quantifies fragmentation of the greenspace into patches in the landscape design map 201. As used herein, patches are separate geometries of greenspace. Patches may be separated by non-greenspace geometries. Alternatively, patches may be separated by a different type of patch. Greenspace fragmentation 243 may be calculated using PD in Table 1.

The greenspace connectedness 245 quantifies connectedness between patches in the landscape design map 201. The COHESION equation of Table 1 may be used to calculate greenspace connectedness 245.

The greenspace aggregation 247 quantifies aggregation of patches in the landscape design map 201. Greenspace aggregation 247 may be calculated with the equation Al in Table 1.

The area weighted mean shape index 249 quantifies irregular shapes. The area weighted mean shape index 249 may be calculated using equation SHAPE_AM in Table 1. The greenspace percentage 251 quantifies a percentage of greenspace in the landscape design map 201 and may be calculated using Equation PLAND in Table 1.

The spatial characteristics 253 may describe quantitative and/or qualitative spatial characteristics of patches and/or groups of patches. The spatial patterns 255 may describe quantitative and/or qualitative spatial patterns of patches and/or groups of patches. The greenspace arrangements 257 may describe quantitative and/or qualitative arrangements of patches and/or groups of patches.

FIG. 2D is a schematic block diagram illustrating one embodiment of the non-communicable disease data 205. In the depicted embodiment, the non-communicable disease data 205 includes at least one of poor mental health 261, heart disease 263, stroke 265, diabetes 267, chronic obstructive pulmonary disease (COPD) 269, physical inactivity 271, emergency visits 273, hospitalizations 275, or other health-related outcomes. The non-communicable disease data 205 may be defined based on available data standards for the adjacent populations.

FIG. 3A is an image of a landscape design map 201. The landscape design map 201 may be generated from a CAD or Photoshop file for a landscape design. The landscape design map 201 may code patch types. In the depicted embodiment, greenspace patches are coded with a dark shading.

FIG. 3B is an image of extracted design characteristics 203. In the depicted embodiment, greenspace patches 246 are extracted from the landscape design map 201 of FIG. 3A. In one embodiment, all other area in the landscape design has a null value.

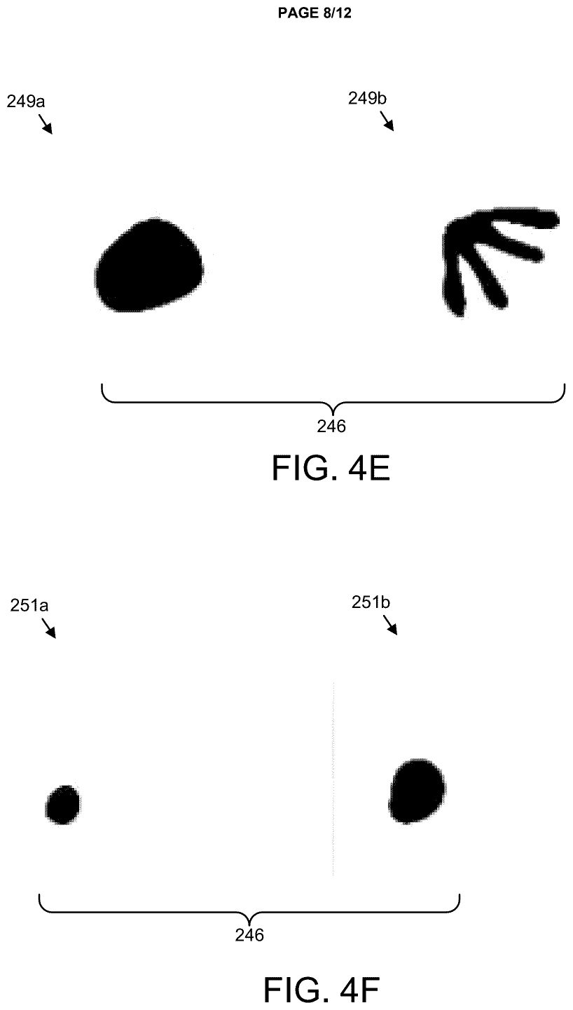

FIGS. 4A-F are images of relative landscape metrics for the greenspace and morphology data 221. The images illustrate lower and higher values of the greenspace and morphology data 221. In the depicted embodiments, the edges are shown as pixilated to support calculations.

FIG. 4A shows patches 246 with lower greenspace mean size 241a and higher greenspace mean size 241b. FIG. 4B shows patches 246 with lower greenspace fragmentation 243a and higher greenspace fragmentation 243b. FIG. 4C shows patches 246 with lower greenspace connectedness 245a and higher greenspace connectedness 245b. FIG. 4D shows patches with a lower aggregation 247a and a higher aggregation 247b. FIG. 4E shows patches 246 with a lower area weighted mean shape index 249a and a higher area weighted mean shape index 249b. FIG. 4F shows patches 246 with a lower greenspace percentage 251a and a higher greenspace percentage 251b.

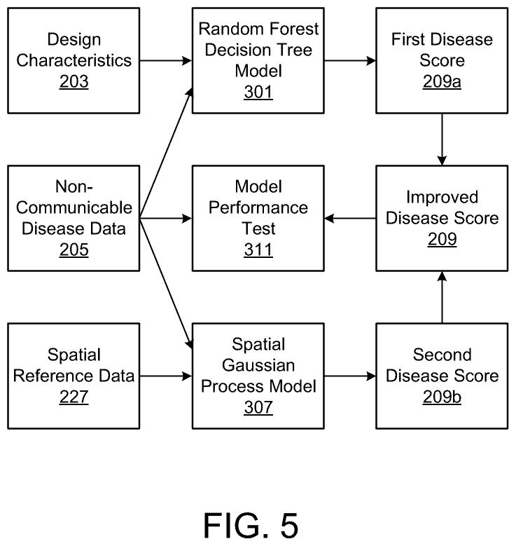

FIG. 5 is a schematic block diagram of one embodiment of a training process for the predictive model 107. In the depicted embodiment, design characteristics 203 and non-communicable disease data 205 are used to train a random forest decision tree model 301. In one embodiment, a portion of the design characteristics 203 are used to train the random forest decision tree model 301. For example, 70 percent of the design characteristics 203 may be used to train the random forest decision tree model 301. The remaining 30% of the design characteristics 203 may be used to set aside data as will be described hereafter. In a certain embodiment, the greenspace and morphology data 221, the demographic data 223, and the geographic data 225 and the non-communicable disease data 205 are used to train the random forest decision tree model 301.

In one embodiment, a spatial Gaussian process model 307 is trained with the spatial reference data 227. A portion of the spatial reference data 227 may be used to train the spatial Gaussian process model 307. In a certain embodiment, 70% of the spatial reference data 227 is used to train the spatial Gaussian process model 307.

In one embodiment, the random forest decision tree model 301 and the spatial Gaussian process model 307 comprise the predictive model 107. The random forest decision tree model 301 may calculate a first non-communicable disease score 209a from design characteristics 203 extracted from at least one landscape design map 201. In addition, the spatial Gaussian process model 307 may calculate the second non-communicable disease score 209b from the Spatial Reference Data 227 extracted from the at least one landscape design map 201 as well as the first non-communicable disease score 209a. The first non-communicable disease score 209a is calculated before the second non-communicable disease score 209b.

The first non-communicable disease score 209a and the second non-communicable disease score 209b may be combined into an improved non-communicable disease score 209. The first non-communicable disease score 209a and the second non-communicable disease score 209b may be summed to generate the improved non-communicable disease score 209. In one embodiment, a weighted average of the first non-communicable disease score 209a and the second non-communicable disease score 209b are summed to generate the improved non-communicable disease score 209.

The non-communicable disease score 209 may estimate a prevalence of at least one of poor mental health 261, heart disease 263, stroke 265, diabetes 267, COPD 269, physical inactivity 271, emergency visits 273, hospitalizations 275, or other health-related outcomes in response to the landscape design of the landscape design map 201. The non-communicable disease score 209 may be for residents in proximity to the implemented landscape design.

The improved non-communicable disease score 209 may be compared with non-communicable disease data 205 corresponding to the landscape design of the landscape design map 201 in the set aside data as part of a model performance test 311.

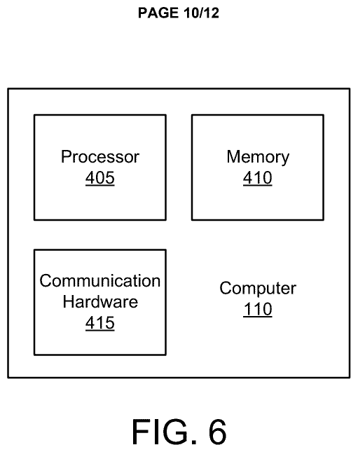

FIG. 6 is a schematic block diagram of one embodiment of the computer 110. In the depicted embodiment, the computer 110 includes a processor 405, a memory 410, and communication hardware 415. The memory 410 may store code and data. The processor 405 may execute the code and process the data. The communication hardware 415 may communicate with other devices and/or networks. In one embodiment, the server 105 is a computer 110.

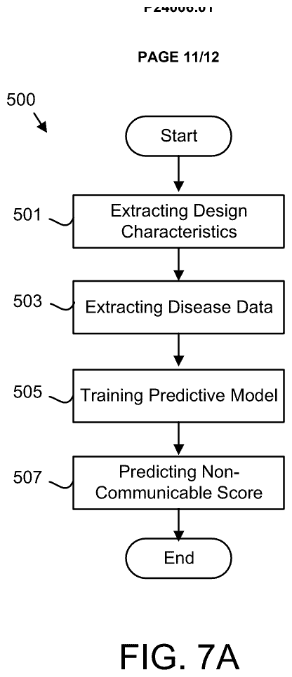

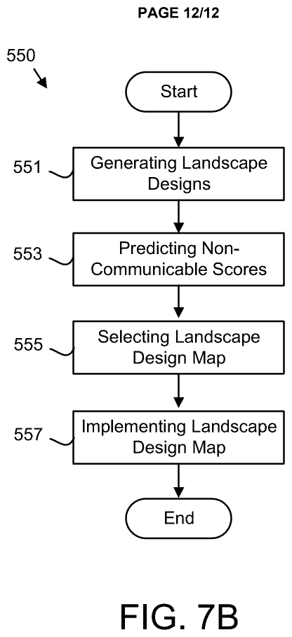

FIG. 7A is a schematic flow chart diagram of an assessment method 500. The method 500 may assess the health effects of landscape designs on adjacent populations. The method 500 may be performed by the assessment system 100 and/or processor 405 of the assessment system 100.

The method 500 may extract 501 the design characteristics 203 from at least one landscape design map 201 of at least one landscape design. In one embodiment, the processor 405 reads a CAD or Photoshop generated image file containing the landscape design map 201 and identifies patches 246 of at least one specified type. For example, the processor 405 may identify grass patches 246, groundcover patches 246, tree patches 246, shrub patches 246, flower patches 246, and the like. The design characteristics 203 for the specified patches 246 are extracted as shown in FIG. 3B. In one embodiment, design characteristics 203 of patches 246 outside of the landscape design are also employed.

The method 500 may extract 503 non-communicable disease data 205 for populations adjacent to the at least one landscape design. In one embodiment, the processor 405 may identify adjacent populations based on the boundaries of the landscape design map 201. For example, the processor 405 may identify populations within the landscape design. Alternatively, the processor 405 may identify population in postal codes the adjacent to the landscape design. The processor 405 may further acquire and extract the non-communicable disease data 205 for hospitals within a hospital buffer of the landscape design.

The method 500 may train 505 a predictive model 107 based on the design characteristics 203 and the non-communicable disease data 205. The predictive model 107 may comprise at least one of a random forest decision tree model 301, the spatial Gaussian process model 307, a Lasso regression model, a Ridge regression model, a support vector machine model, an ensemble tree model, a logistic regression model, a k-means model, a linear regression model, a nonlinear regression model, a decision tree model, a generalized additive model, a neural network model, a naïve Bayes model, a discriminant analysis model, a k-nearest neighbor model, or other data science methods, or combinations thereof. The processor 405 may train 505 the predictive model 107 using the design characteristics 203 and the non-communicable disease data 205 as described in FIG. 5.

The method 500 predicts 507 the non-communicable disease score 209 for a given landscape design map 201 and the method 500 ends. In one embodiment, the given landscape design map 201 is not used to train 505 the predictive model 107. The design characteristics 203 and the non-communicable disease data 205 may be extracted for the given landscape design map 201. The non-communicable disease score 209 may estimate the prevalence of at least one of poor mental health 261, heart disease 263, stroke 265, diabetes 267, COPD 269, physical inactivity 271, emergency visits 273, hospitalizations 275, and/or other health-related outcomes for the adjacent population to the landscape design.

FIG. 7B is a schematic flow chart diagram of an implementation method 550. The implementation method 550 may generate, select, and/or implement a given landscape design map 201 from a plurality of landscape design maps 201. The implementation method 550 may be performed by the assessment system 100 and/or processor 405 of the assessment system 100.

The method 550 may generate 551 at least two landscape design maps 201. The landscape design maps 201 may be algorithmically generated. Alternatively, the landscape design maps 201 may be generated by at least one designer. In a certain embodiment, the landscape design maps 201 are generated 551 by a plurality of submitters.

The method 550 may predict 553 non-communicable disease scores 209 for each of the landscape design maps 201. In one embodiment, the processor 405 extracts design characteristics 203 for each landscape design map 201 for the landscape design site and uses the non-communicable disease data 205 to generate the non-communicable disease scores 209.

The method 550 may select 555 a given landscape design map 201 based on the non-communicable disease scores 209. In one embodiment, a landscape design map 201 with the best non-communicable disease score 209 is selected as the given landscape design map 201.

The method 550 may further implement 557 the given landscape design map 201. Because the given landscape design map 201 is selected based on the communicable disease score 209, the implemented landscape design is more effective in promoting good health for an adjacent population. As a result, the efficiency and effectiveness of landscape design and implementation and the assessment system 100 is improved.

This description uses examples to disclose the invention and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method comprising:
extracting design characteristics from at least one landscape design map;
extracting non-communicable disease data for adjacent populations to the at least one landscape design map;
training a predictive model based on the design characteristics and the non-communicable disease data; and
predicting a non-communicable disease score for a given landscape design map from the predictive model.

2. The method of claim 1, the method further comprising:
generating at least two landscape design maps;
predicting non-communicable disease scores for the at least two landscape design maps;
selecting the given landscape design map from the at least two landscape design maps based on the non-communicable disease scores; and
implementing the given landscape design map.

3. The method of claim 1, wherein the design characteristics comprise at least one of greenspace and morphology data, demographic data, geographic data, spatial reference data.

4. The method of claim 3, wherein the greenspace and morphology data comprises at least one of a greenspace mean-size, a greenspace fragmentation, greenspace connectedness, greenspace aggregation, an area-weighted mean shape index, a greenspace percentage, or alternative metrics that characterize greenspace spatial features, patterns or arrangements.

5. The method of claim 1, wherein the predictive model comprises at least one of a random forest decision tree model and a spatial Gaussian process model.

6. The method of claim 5, wherein the random forest decision tree model of the predictive model generates a first non-communicable disease score and the spatial Gaussian process model generates a second non-communicable disease score and the first non-communicable disease score and the second non-communicable disease score are combined to generate the non-communicable disease score.

7. The method of claim 1, wherein the non-communicable disease score estimates prevalence of at least one of poor mental health, heart disease, stroke, diabetes, chronic obstructive pulmonary disease (COPD), physical inactivity, emergency visits, and hospitalizations.

8. The method of claim 1, wherein the predictive model further comprises at least one of a Lasso regression model a Ridge regression model, a support vector machine model, an ensemble tree model, a logistic regression model, a k-means model, linear a regression model, a nonlinear regression model, a decision tree model, a generalized additive model, a neural network model, a naïve Bayes model, a discriminant analysis model, and a k-nearest neighbor model.

9. An apparatus comprising:
a processor executing code stored in a memory to perform:
extracting design characteristics from at least one landscape design map;
extracting non-communicable disease data for adjacent populations to the at least one landscape design map;
training a predictive model based on the design characteristics and the non-communicable disease data; and
predicting a non-communicable disease score for a given landscape design map from the predictive model.

10. The apparatus of claim 9, the processor further:
generating at least two landscape design maps;
predicting non-communicable disease scores for the at least two landscape design maps;
selecting the given landscape design map from the at least two landscape design maps based on the non-communicable disease scores; and
implementing the given landscape design map.

11. The apparatus of claim 9, wherein the design characteristics comprise at least one of greenspace and morphology data, demographic data, geographic data, and spatial reference data.

12. The apparatus of claim 11, wherein the greenspace and morphology data comprises at least one of a greenspace mean-size, a greenspace fragmentation, greenspace connectedness, greenspace aggregation, an area-weighted mean shape index, a greenspace percentage, or alternative metrics that characterize greenspace spatial features, patterns or arrangements.

13. The apparatus of claim 9, wherein the predictive model comprises at least one of a random forest decision tree model and a spatial Gaussian process model.

14. The apparatus of claim 13, wherein the random forest decision tree model of the predictive model generates a first non-communicable disease score and the spatial Gaussian process model generates a second non-communicable disease score and the first non-communicable disease score and the second non-communicable disease score are combined to generate the non-communicable disease score.

15. The apparatus of claim 9, wherein the non-communicable disease score estimates prevalence of at least one of poor mental health, heart disease, stroke, diabetes, chronic obstructive pulmonary disease (COPD), physical inactivity, emergency visits, and hospitalizations.

16. A computer readable storage medium storing non-transitory computer readable code executable by a processor to perform:
extracting design characteristics from at least one landscape design map;
extracting non-communicable disease data for adjacent populations to the at least one landscape design map;
training a predictive model based on the design characteristics and the non-communicable disease data; and
predicting a non-communicable disease score for a given landscape design map from the predictive model.

17. The computer readable storage medium of claim 16, the processor further:
generating at least two landscape design maps;
predicting non-communicable disease scores for the at least two landscape design maps;
selecting the given landscape design map from the at least two landscape design maps based on the non-communicable disease scores; and
implementing the given landscape design map.

18. The computer readable storage medium of claim 16, wherein the design characteristics comprise at least one of greenspace and morphology data, demographic data, geographic data, and spatial reference data.

19. The computer readable storage medium of claim 18, wherein the greenspace and morphology data comprises at least one of a greenspace mean-size, a greenspace fragmentation, greenspace connectedness, greenspace aggregation, an area-weighted mean shape index, a greenspace percentage, or alternative metrics that characterize greenspace spatial features, patterns or arrangements.

20. The computer readable storage medium of claim 16, wherein the predictive model comprises at least one of a random forest decision tree model and a spatial Gaussian process model.
